(19) United States
(12) Patent Application Publication
(10) Pub. No.: US 2003/0083227 A1
(43) Pub. Date: May 1, 2003

(54) Novel combination therapy to treat glaucoma

(76) Inventors: Mortimer M. Civan, Wynnewood, PA (US); Kenneth A. Jacobson, Silver Spring, MD (US); Anthony D.C. MacKnight, Dunedin (NZ); Claire H. Mitchell, Philadelphia, PA (US); Richard A. Stone, Havertown, PA (US)

Correspondence Address: Evelyn H. McConathy, Esquire, Dilworth Paxson LLP, 3200 Mellon Bank Center, 1735 Market Street, Philadelphia, PA, 19103, US

(21) Appl. No.: 10/217,755

(22) Filed: Aug. 13, 2002

Related U.S. Application Data
(63) Continuation-in-part of application No. 10/009,581, filed on Apr. 30, 2002.
(60) Provisional application No. 60/133,180, filed on May 7, 1999.
(60) Provisional application No. 60/312,036, filed on Aug. 13, 2001.

(51) Int. Cl. A61K 31/00

(52) U.S. Cl. 514/1

(57) ABSTRACT

Provided is a method for modulating, controlling or regulating intraocular pressure and secretion of the aqueous humor of the eye, in particular for treating or reducing elevated intraocular pressure or secretion, e.g., related to glaucomas. Selected combined drug therapy effectively and synergistically modulates intraocular pressure by either (1) double-blocking the uptake step, wherein both transporters in the first (entry step) of aqueous humor formation are blocked or inhibited; or (2) blocking the entry and exit steps, wherein the sodium-hydrogen (Na⁺/H⁺) exchanger underlying the entry step is blocked or inhibited, and also lowering or reducing the activity of the chloride (Cl⁻) channels involved in the second (exit) step of aqueous humor formation. By combining the selected drugs or compounds to produce a combined or synergistic modulating effect, control of IOP is achieved at very low concentrations, with fewer adverse side-effects on the patient. Moreover, the selectivity of each component in the combination permits the fluid levels in the intraocular space to be tailored to the individual patient or circumstance.

### REFERENCE TO RELATED APPLICATIONS

This application is a Continuation-in-Part Application of U.S. Application No. 10/009,581, filed Apr. 30, 2002, claiming priority to International filing date May 8, 2000, which claims priority to U.S. Provisional Applications No. 60/133,180, filed May 7, 1999; it also claims the benefit of U.S. Provisional Application No. 60/312,036, filed Aug. 13, 2001. The priority dates of each application are herein claimed, and the content of each application is herein incorporated by reference.

### GOVERNMENT INTERESTS

This invention was supported in part by Grant Nos. EY05454, EY08343, EY10691, EY12213, EY13624 and EY01583 from the U.S. National Institutes of Health, and by National Heart, Lung and Blood Institute HL-07027. The Government may have certain rights in this invention.

### FIELD OF THE INVENTION

The present invention relates to the field of ophthalmology. In particular, the invention relates to the prevention and treatment of glaucoma and associated elevations of intraocular pressure.

### BACKGROUND OF THE INVENTION

Glaucomas are a group of blinding diseases highly prevalent throughout the world, currently affecting an estimated three million people in the United States, with 300,000 new cases diagnosed every year. Glaucoma results from obstructed outflow from the aqueous humor of the eye, resulting in elevated intraocular pressure in the anterior chamber, and visual loss attributed to progressive damage of the optic nerve, and consequent loss of retinal ganglion cells (Quigley et al., Invest. Ophthalmol. Vis. Sci. 19:505 (1980)). Increase of the intraocular pressure (“IOP”) of the eye is the major, and best understood, risk factor for the appearance and progression of glaucomatous optic neuropathy. Elevated or increased intraocular pressure (“IOP”) can also be caused by other conditions, such as impaired intraocular fluid transport caused by eye surgery, including surgery for glaucoma. The IOP, itself, reflects a balance between the rates of inflow (fluid formation) and outflow (fluid return) of the aqueous humor by re-absorption. Medical approaches to treating glaucoma are frequently directed at reducing the rate of net formation of aqueous humor.

The aqueous humor of the eye is formed by the ciliary epithelium, comprising two cell layers, whose apical membranes are juxtaposed. The outer pigmented ciliary epithelial (PE) cells face the stroma, while the inner non-pigmented ciliary epithelial (NPE) cells are in contact with the aqueous humor. Secretion involves primary solute transfer, primarily NaCl, with accompanying water movement, from the blood or supporting stroma, across the basolateral membranes of the PE cells into the aqueous humor in the contralateral posterior chamber of the eye (Cole, Exp. Eye Res. 25(Suppl):161-176 (1977)). This provides an osmotic driving force for the secondary osmotic transfer of water down its chemical gradient, although a more direct coupling between water and solute may also proceed across the epithelia (Meinild et al., J. Physiol. 508:15-21 (1998)).

The secretion of aqueous humor into the eye results as a consequence of two opposing physiological processes: fluid secretion into the eye by the NPE cells and fluid reabsorption (secretion out of the eye) by the PE cells. Thus, both release of chloride ions by the NPE cells into the adjacent aqueous humor enhance secretion, and chloride ion release by the PE cells into the neighboring stroma reduce net secretion (Civan, Current Topics in Membranes 45:1-24 (1998), Tripathi, In: The Eye, Chap. 3, pp 163-356, Davson & Graham (eds), Academic Press, New York, (1974)). Intraocular pressure reflects a balance between the rates of secretion and outflow of the aqueous humor.

A major factor governing the rate of secretion is the rate of chloride ion (Cl⁻) release from the NPE cells into the aqueous humor (Civan, News Physiol. Sci. 12:158-162 (1997)). Thus, the activity of the Cl⁻ channels is a rate-limiting factor in aqueous humor secretion, given the low baseline level of channel activity and the predominance of the chloride anion in the transferred fluid (Coca-Prados et al., Am. J Physiol. 268:C572-C579 (1995)). Adenosine has been shown to activate NPE Cl⁻ channels that subserve this release (Carre et al., Am. J. Physiol. 273 (Cell Physiol. 42) C1354-C-1361 (1997)). Adenosine triggered isotonic shrinkage of cultured human cells from the human ciliary epithelial (HCE) cell line. In addition, adenosine produced a Cl⁻-dependent increase in short circuit current across rabbit iris ciliary body while the non-metabolizable adenosine analog 2-Cl-adenosine was shown to activate Cl⁻ currents in HCE cells using the whole patch-clamp technique. However, since the concentrations of agonist used by Carre et al., 1997 were capable of stimulating all four known adenosine receptor subtypes found in ciliary epithelial cells: A₁, A_{2A}, A_{2B} and A₃, the effect on Cl⁻ channels in NPE cells remained unknown. It was not until the study by Mitchell et al. (Am. J Physiol. 276 (Cell Physiol. 45) C659-C-666 (1999)) that it was determined that A₃ receptors are present on both rabbit and human NPE cells and underlie the activation of NPE Cl⁻ channels by adenosine (see also Carre et al., Am. J. Physiol. Cell Physiol. 279:C440-C451 (2000)).

Structurally the mouse eye parallels the aqueous humor outflow pathways in the human and shows similar functional responses to drugs that inhibit aqueous humor inflow and facilitate outflow in the human. Thus, the mouse is a particularly suitable non-primate model for studying the genetic control of physiological and pharmacological function. However, the anterior chamber of a mouse eye contains only about 2-4 μl of aqueous humor, which until recently, complicated efforts to measure IOP in the mouse reliably.

However, the adaptation of the servo-null micropipette system (SNMS) by Avila et al., as reported in Invest. Ophthalmol. Vis. Sci. 42:1841-1846 (2001A), has overcome the difficulties previously encountered in measuring the IOP in such small eyes, e.g., in the mouse, thereby permitting reliable monitoring over periods as long as 45 minutes. Using this technique, Avila et al. were able to measure IOP responses to subtype-specific adenosine A₃ receptor (AR) agonists and antagonists in the mouse (Brit. J. Pharmacol. 134:241-245 (2001B)), and found that they increased and decreased IOP respectively, consistent with the in vitro findings. Additionally, the investigators measured mouse IOP responses to A1 and A2A agonists and antagonists, which proved consistent with earlier findings in rabbits and monkeys. This confirmed that the mouse eye was a reliable model for IOP of the human eye. Moreover, a large increase in mouse IOP triggered by applied adenosine was largely blocked and prevented by a pre-application of A₃AR antagonists. When studied in A₃AR^{-/-} knockout mice, the reduced IOP and altered purigeneric responses of IOP supported the conclusion that A₃ARs contribute to the regulation of IOP in vivo (Avila et al., Invest. Ophthalmol. Vis. Sci. 43:in press (2002)).

FIG. 1 depicts a minimalist, and necessarily incomplete, consensus model of aqueous humor secretion from Avila et al., Invest. Ophthalmol. Vis. Sci. 43:1897-1902 (2002) (Carré et al., Curr. Eye Res. 11:609-624 (1992); Chu et al., Invest. Ophthalmol. Vis. Sci. 28:445-450 (1987); Wolosin et al., Exp. Eye. Res. 64:945-952 (1997)). “Inflow,” the transfer of fluid from body side or “stromal side” into the aqueous humor, is presented as basically a 3-step process. First, as shown, water and salt, NaCl, is initially taken up from the stroma into the pigmented ciliary epithelial (PE) cells, supported by paired Na⁺/H⁺ and Cl⁻/HCO₃⁻ antiports, and the Na⁺-K⁺-2Cl⁻ symport (Kaufman et al., In: Textbook of Ophthalmology, Vol. 7, Podos & Yanoff (eds), Mosby, St Louis, pp 9.7-9.30 (1994); McLaughlin et al., Invest Ophthalmol. Vis. Sci. 39:1631-1641 (1998), Walker et al., Am. J Physiol. 276:C1432-1438 (1999); Wiederholt et al., In: Carbonic Anhydrase, Botré, Gross, Storey (eds), VCH, New York, pp 232-244 (1991); Edelman et al., Am. J Physiol. 266:C1210-C1221 (1994); Wiederholt et al., Pflügers Arch. 407(Suppl. 2):S112-S115 (1986)).

Second, the salt and water from the PE cells diffuses across the gap junctions into the second cell layer [non-pigmented ciliary epithelial (NPE) cells] abutting the aqueous humor (Coca-Prados et al., Curr. Eye Res. 11:113-122 (1992); Edelman et al., 1994; Mitchell et al., FASEB J 11:A301 (1998); Oh et al., Invest. Ophthalmol. Vis. Sci. 35:2509-2514 (1994); Raviola et al., Invest. Ophthalmol. Vis. Sci. 17:958-981 (1978); Walker et al., 1999; Wolosin et al., In: The Eye's Aqueous Humor: From Secretion to Glaucoma, Civan (ed), Academic Press, Boston, pp 135-162 (1998)).

Finally, the salts and fluids are released into the aqueous humor by the contiguous NPE cells through the Na⁺, K⁺-activated ATPase exchange pump and Cl⁻ channels (Jacob et al., Am. J. Physiol. 271:C703-C720 (1996); Civan, 1997). Using several in vitro preparations [freshly harvested bovine NPE cells (Carre et al., 1997), cultured human NPE cells (Carre et al., 1997, 2000, Mitchell et al., Am. J Physiol. 276 (Cell Physiol. 45):C659-C666 (1999)), and rabbit iris ciliary body (Carre et al., 1997, Mitchell et al., 1999)], it has been shown that agonists of A₃-subtype adenosine receptors activate the Cl⁻ channels of NPE cells.

The uptake step into the PE cells is largely electroneutral, although the underlying mechanism is not fully known. However, electron probe X-ray microanalyses (McLaughlin et al., 1998) of excised intact rabbit iris-ciliary bodies, support the concept that the predominant uptake mechanism underlying baseline physiologic conditions is the paired antiports. Indeed, the paired antiports can so elevate the intracellular Cl⁻ level as to favor the cellular release of NaCl through the Na⁺-K⁺-2Cl⁻ symport.

Current treatment methods to relieve intraocular pressure include forming small laser penetrations in the eye to release excess pressure (e.g., trabeculectomy), as well as the use of systemic and topical drugs for lowering intraocular pressure. At the present time, medical control of intraocular pressure and glaucoma consists of topical, oral or intravitreous administration of many compounds. See generally, Horlington, U.S. Pat. No. 4,425,346; Komuro et al., U.S. Pat. No. 4,396,625; Gubin et al., U.S. Pat. No. 5,017,579; Yamamori et al., U.S. Pat. No. 4,396,625; Abelson, U.S. Pat. No. 4,981,871; and Bodor et al., U.S. Pat. No. 4,158,005.

Among the most effective medical therapies for glaucoma are strategies aimed at reducing intraocular pressure by reducing the net rate of aqueous humor formation by the ocular ciliary epithelial bilayer (see generally, Shields, Textbook of Glaucoma, 3rd Ed., Williams & Wilkins, Baltimore (1992)). This can occur either by blocking unidirectional secretion from stroma to the aqueous humor or by stimulating flow in the opposite direction (Caprioli et al., Yale J. Biol. Med. 57:283-300 (1984); Civan et al., Exp. Eye Res. 62:627-640 (1996)).

Four primary classes of drugs are used to treat glaucoma. These include: miotics (e.g., pilocarpine, carbachol and acetylcholinesterase inhibitors); sympathomimetics (e.g., epinephrine, metipranolol, dipivefrin, carbachol, dipivalyl, and parn-aminoclonidine); beta-blockers (e.g., betaxolol, levobunolol and timolol) and potent cholinesterase inhibitors (e.g., echothiophate); and carbonic anhydrase inhibitors (e.g., acetazolamide, methazolamide, dorzolamidet and ethoxzolamide). For example, miotics and sympathomimetics are believed to lower intraocular pressure by increasing the outflow of aqueous humor, while beta-blockers and carbonic anhydrase inhibitors are believed to operate by decreasing the formation of aqueous humor (Ritch et al., (1996) In: The Glaucomas (eds Ritch, Shields, Krupin) 2nd ed., pp. 1507-1519, Mosby, St. Louis). The non-selective, topical, β- and β₁-adrenergic antagonists have proven to be useful for lowering the secretory rate of fluids in the eye (aqueous humor inflow), and thereby for controlling intraocular pressure (Gieser et al., (1996) In: The Glaucomas, supra, pp. 1425-1448). Timolol reportedly binds to β-adrenergic receptors of the ciliary processes with high affinity (Vareilles et al., Invest. Ophthalmol. Vis. Sci. 16:987-996 (1977)), and is among the most widely used and effective drugs for lowering the intraocular pressure of glaucomatous patients (Gieser et al., 1996). Another new type of drug, precursor prostaglandin compounds (e.g., latanoprost), which enhance outflow are also in current use.

Nevertheless, each of the known drugs in current use is accompanied by significant adverse, systemic side-effects, even when administered topically, and inconvenient dosing schedules, which may lead either to decreased patient compliance or to termination of therapy. Miotics tend to reduce the patient's visual acuity, particularly in the presence of lenticular opacities. Topical beta blockers, such as timolol, have been associated with side-effects such as fatigue, confusion, or asthma; while exacerbated cardiac symptoms have been reported after rapid withdrawal of topical beta blockers. Oral administration of carbonic anhydrase inhibitors, such as acetazolamide, while useful, have been associated with systemic side effects including chronic metabolic acidosis.

Accordingly, because of the insidious nature of glaucomas and other conditions affecting the intraocular pressure in the eye and the difficulties in treating them, there has been an on-going and long-felt need in the art for the development of methods for the safe and reliable prevention, control or treatment of elevated intraocular pressure, that can be utilized before significant damage to the optical nerve occurs. Also needed is the discovery of compositions or therapies that will cause fewer or reduced adverse side-effects when compared to present drugs, or methods that will permit known (or yet to be discovered) drugs at lower dosages that will permit the drugs to be used without adverse effects.

Lower than normal intraocular pressure can also be problematic, caused for example, by a variety of conditions, such as surgery for glaucoma, retinal detachment, uveitis, and the like. However, since no drugs are presently available for the safe and effective prevention, modulation or regulation of reduced intraocular pressure without adverse side-effects, there remains a need for the development of more effective treatment methods for surgically-induced low or depressed intraocular pressure, as well as elevated intraocular pressure.

### SUMMARY OF THE INVENTION

The present invention, therefore, meets a particular need in the art by providing methods for preventing, modulating or regulating intraocular pressure, in particular for treating or reducing elevated intraocular pressure. Specifically, the present invention provides combined therapeutic methods by which intraocular fluid pressure can be selectively and reversably increased, decreased, or maintained at a predetermined level, although primarily the invention will be useful to relieve or prevent elevated levels of intraocular fluid in, for example, glaucoma patients, before vision is adversely and permanently affected. In addition, the present combined therapeutic methods permit known compounds to be used in such low dosages as to permit effective modulation of IOP with little or no adverse side-effects.

The present invention provides several methods for regulating, controlling or modulating aqueous humor secretion, comprising the step of administering to ciliary epithelial cells of the aqueous humor, an effective (“secretion-modulating”) amount of more than one pharmaceutical compositions administered in combination (or sequentially, but in sufficiently close proximity of time as to achieve a combined effect). Further provided is in vivo evidence that the combinations of drugs or therapeutic moieties effectively and synergistically lower intraocular pressure (IOP) by: (1) double-blocking the uptake step, wherein both transporters in the first (entry step) of aqueous humor formation (the paired Na⁺/H⁺ and Cl⁻/HCO₃⁻ antiports and the Na⁺-K⁺-2Cl⁻ symport) are blocked or inhibited; or (2) blocking or inhibiting the entry and the exit steps, wherein the sodium-hydrogen (Na⁺/H⁺) exchanger underlying the entry step is blocked and also the activity is lowered or reduced of the chloride (Cl⁻) channels involved in the second (exit) step of aqueous humor formation.

The combined compositions are administered either in actual combination or sequentially to the patient in closely timed dosages, which are sufficient to produce a combined and preferably synergistic effect in the patient, thereby modulating, preferably by blocking or inhibiting elevated IOP. In fact, when either the secretion into the aqueous humor cells is elevated, or the fluid pressure or intraocular pressure is elevated in a patient, the drugs in the combination therapy are administered in a combined amount, that is sufficient to reduce the elevated secretion. Moreover, the modulating effect is reversible when the combination therapy ceases.

In addition, methods are provided wherein the modulator combination is administered to the cells in vitro or in vivo. The latter methods offer regulation, control or modulation of fluid pressure or intraocular pressure in an individual or subject.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

In the following Figures, and in the Examples from which they are derived, values are presented as the means ±1 SE. The number of experiments is indicated by the symbol n or N.

FIG. 1 depicts a consensus model of aqueous humor formation and NaCl secretion by the ciliary epithelium. Carbonic anhydrase limited delivery of H⁺ and HCO₃⁻ limits uptake of stromal NaCl through paired antiports. In parallel, NaCl can also enter (or exit) PE cells through the Na⁺-K⁺-2Cl⁻ symport. At the contralateral surface, Na+ and Cl⁻ can be released from the NPE cells into the aqueous humor through Na⁺, K⁺-activated ATPase and Cl⁻ channels, respectively.

FIGS. 2A-2C graphically depict the effect of 4,4′-diisothiocyanatostilbene-2,2′-disulfonic acid (DIDS) and bumetanide on the regulatory volume increase (RVI). Neither bumetanide (FIG. 2A, N=9), nor DIDS (FIG. 2B, N=3) inhibited the volume recovery, but the two inhibitors together blocked the RVI (FIG. 2C, N=8, P<0.05).

FIGS. 3A and 3B graphically depict the responses of mouse IOP to inhibition of Na^{+/}H⁺ antiports with DMA or to inhibition of Na⁺-K⁺-2Cl⁻ symport with bumetanide. (FIG. 3A) DMA (1 mM, 2.94 μg) lowered IOP. (FIG. 3B) Neither 1 mM(3.64 μg) nor 10 mM (36.4 μg) bumetanide by itself significantly altered mouse IOP.

FIGS. 4A-4D graphically depict responses to combined (sequential) topical addition of direct or indirect inhibitors of Na⁺/H⁺ antiports, followed by bumetanide: (FIG. 4A) 1 mM (2.94 μg) DMA followed by 1 mM (3.64 μg) bumetanide; (FIG. 4B) 1 mM (5.34 μg) BIIB723 followed by 1 mM (3.64 μg) bumetanide; (FIG. 4C) 55.4 mM (200 μg) dorzolamide followed by 1 mM (3.64 μg) bumetanide; and (FIG. 4D) 1 mM EIPA (3.00 μg) followed by 1 mM (3.64 μg) bumetanide. In each case, bumetanide significantly reduced IOP after prior inhibition of the Na^{+/}H⁺ antiport.

FIG. 5 graphically depicts the effects of acetazolamide and intraperitoneal water on the IOP of an A₃^{-/-} mouse. Intraperitoneal acetazolamide lowered IOP and subsequent intraperitoneal water loading elicited an expected increase in IOP.

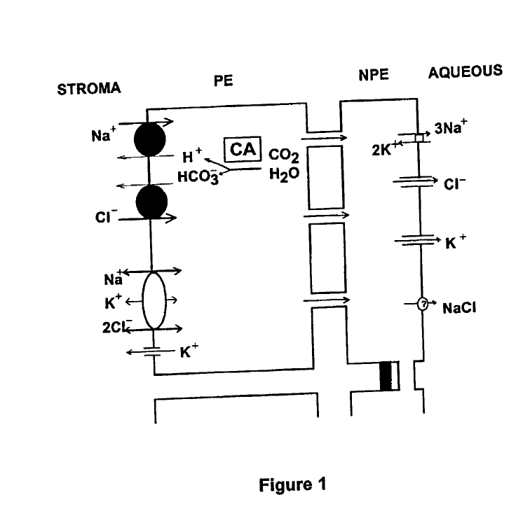

### DETAILED DESCRIPTION OF EMBODIMENTS OF THE INVENTION

The methods and compositions of the present invention are intended for treatment of glaucoma and other conditions, which manifest elevated intraocular pressure in the eye of a patient, particularly human patients, but also including other mammalian hosts. Glaucoma is a term which embraces a group of ocular diseases characterized by elevated intraocular pressure levels which can damage the eye, and destroy the optic nerve and related ganglia. In addition, normotensive glaucoma is characterized by an apparent nonelevated intraocular pressure. However, for the patient suffering from normotensive glaucoma, the apparently normal pressure is sufficiently high for that particular patient as to cause the same types of nerve and vision damage as elevated pressure would cause in patients with other glaucomas.

Therefore, the glaucomas treated by the methods of the present invention are not limited exclusively to elevated intraocular pressure. Other conditions which result in elevated intraocular pressure levels include cataract surgery, steroid treatment, and treatment with other drugs known to cause intraocular pressure. The methods and compositions of the present invention are intended to treat all such conditions, preferably to lower the intraocular pressure to a manageable and safe level. Moreover, the methods are also effective in the treatment of lower than normal intraocular pressure levels.

The present invention provides in vivo evidence that combinations of drugs or therapeutic moieties (the “combined modulator”) effectively and synergistically lower intraocular pressure (IOP) by either: (1) double-blocking of uptake step, wherein both transporters in the first (entry step) of aqueous humor formation are blocked or inhibited; or (2) blocking of the entry and exit steps, wherein the sodium-hydrogen (Na⁺/H⁺) exchanger underlying the entry step is blocked and also the activity is lowered or reduced of the chloride (Cl⁻) channels involved in the second (exit) step of aqueous humor formation. These discoveries, which are discussed in detail below, permit strategies to be developed to use drugs at very low, focussed concentrations for preventing, modulating or regulating intraocular pressure, most particularly for treating or reducing elevated intraocular pressure.

In the normal PE/NPE cell bilayer, water and small non-polar molecules would typically cross rapidly. However, charged molecules and salts cross the cell barrier through carrier transmembrane proteins. Some carrier proteins (“uniports”) simply transport a single solute from one side of the cell layer to the other. Others function as coupled transporters, in which the transfer of one solute depends upon the simultaneous or sequential transfer of a second solute, either in the same direction (a “symport”), or in the opposite direction (an “antiport”). Many active transport systems are driven by the energy stored in ion gradients, some of which function as symports, others as antiports. Two important examples of ion gradients used to drive an antiport system are the antiports that function together to regulate intracellular pH in many animals.

Almost all vertebrate cells have a NA⁺ driven antiport, called an “Na⁺/H⁺ exchange carrier” or “exchanger,” which plays a crucial role in maintaining intracellular pH (“pHi,” usually around 7.1 or 7.2). This carrier couples the efflux of H⁺ to the influx of Na⁺, and thereby removes excess H⁺ ions produced as a result of the acid-forming reactions in the cell. Thus, the Na⁺/H⁺ exchanger regulates pHi. At higher pHi, the exchanger is inactive, but activity increases as the pHi becomes more acid.

The “Cl⁻/HCO₃ exchanger,” like the Na⁺/H⁺ exchanger, regulates pHi, but in the opposite direction. Its activity increases as pHi rises, increasing the rate at which HCO₃⁻ (also referred to as bicarbonate) is ejected from the cell in exchange for Cl⁻, thereby decreasing pHi. Flow through the exchangers is driven by the electrochemical gradient for the ion.

Double Blocking the Uptake Step

The basis for the first step in inflow into the aqueous humor, uptake of salt into the PE-cell layer, has been the subject of considerable controversy. Some investigators have reported that the Na⁺-K⁺-2Cl⁻ co-transporter (or symport) is primarily involved. Others have concluded that the parallel operation of Na⁺/H⁺ and Cl⁻/HCO₃⁻ exchangers (or antiports) is primarily responsible. Previous reports by the inventors have identified and characterized the sodium/proton exchanger (or “antiport”) and determined its important role in the first step, including the uptake of fluids and salts into the PE cells (U.S. patent application S. No. 10/009,581, herein incorporated by reference teaches that both paired Na⁺/H⁺ and Cl⁻/HCO₃⁻ antiports and the Na⁺-K⁺-2Cl⁻ symport are involved in net uptake).

In the present invention, the inventors have confirmed in vivo, by studying IOP in a live mouse, the earlier findings that identified in vitro (in cultured bovine PE cells and RNA preparations of human ciliary body) the molecular basis for the paired antiport activity of the NHE-1 Na⁺/H⁺ exchanger, and the AE2 Cl⁻/HCO₃⁻ exchanger. (Counillon et al., Pflugers Arch. (Eur. J. Physiol.) 440:667-678 (2000); Avila et al., 2001A). Because the NHE-1 exchanger is highly sensitive to several blockers of the sodium/proton symport, it was possible to selectively block the exchangers specifically involved in aqueous humor inflow.

An electron-probe X-ray microanalysis (McLaughlin et al., 1998) of excised rabbit ciliary epithelium indicated that the paired antiports provide the dominant entry pathway under physiological conditions, and further suggested that carbonic anhydrase inhibitors (commonly used to treat glaucoma) act by blocking Na⁺/H⁺ exchange. More recently an electron-probe X-ray microanalysis (McLaughlin et al., Am. J. Physiol. Cell Physiol. 281:C865-C875(2001)) further suggested that another very widely used antiglaucomatous drug (timolol) acts primarily in the same way, blocking Na⁺/H⁺ exchange.

More importantly, however, in the present invention, the inventors have determined that blocking the Na⁺-K⁺-2Cl⁻ co-transporter with bumetanide had no significant effect on IOP in the mouse (N=8, 10 uM-10 mM concentrations in topical drops), as also observed in monkeys (Gabelt et al., Invest. Ophthamol. Vis. Sci. 38:1700-1707 (1997). However, in a preferred and exemplified embodiment of the invention, after applying a topical inhibitor (ethylisopropyl-amiloride) of Na⁺/H⁺ antiport exchange, the administration of 10 mM bumetanide reduced IOP by 4.0±0.6 mm Hg (mean±SE, N=6, P<0.01). By comparison, the same concentration of bumetanide alone produced no significant change in 4 mice (−1.0±1.1 mm Hg). Thus, it was demonstrated that by blocking both known ports of NaCl entry into the ciliary epithelium (the paired antiports and the symport) a synergy results, such that IOP is reduced in mammals, including primates and humans, much more effectively than reductions achieved by treatment with currently applied drugs that produce only a single effect.

Blocking the Entry and Exit Steps

The basis of the release step of solute and water into aqueous humor is generally via extrusion of Na⁺ through the Na⁺, K⁺-activated ATPase and the release of Cl⁻ through the Cl⁻ channels. Agonists of A₃-subtype adenosine receptors have been found to activate the Cl⁻ channels of NPE cells. This action enhances aqueous humor inflow and raises IOP.

Conversely, antagonists of A₃-subtype adenosine receptors have been shown to lower IOP (Avila et al., Invest. Ophthalmol. Vis. Sci. 43:in press (2002)). Use of A₃ antagonists is particularly encouraging since mice with selective knockout of the A₃-receptor gene (leaving the A₁, A_{2A} and A_{2B} receptors intact) display normal behavior (Salvatore, et al., J. Biol. Chem. 275(6):4429-4434 (2000), Tilley et al., J. Clin. Invest. 105:361-367 (2000)). Therefore, it was concluded that pharmacologic inhibition of the A₃-subtype adenosine receptor would produce few side-effects.

In light of the foregoing, a preferred embodiment of the present invention provides an alternative combinatorial drug approach for more effectively controlling IOP, wherein both the first step of aqueous humor formation (entry into the ciliary epithelium) and the release step of the chloride ions from the aqueous humor are simultaneously blocked. The advantage of this approach is that each of the two steps can be selectively targeted, thereby reducing the likelihood of troublesome side-effects. As discussed with regard to the embodiment above, in which the two entry steps were blocked, the NHE-1 exchanger can be selectively blocked, which is important in the first step of aqueous humor formation. However, it is also possible to block activation of the final step of aqueous humor formation by applying A₃-subtype adenosine-receptor antagonists. Therefore, by administering both classes of drugs together, the effect is highly advantageous (blocking or controlling both the first and the final steps of aqueous humor formation), resulting in an efficacious mechanism for modulating IOP that is also relatively free of side effects.

Elevated intraocular pressures often exceed 20 mm Hg and it is desirable that such elevated pressures be lowered to below 18 mm Hg. In the case of low-tension glaucoma, it is desirable for the intraocular pressure to be lowered below that exhibited by the patient prior to treatment. Intraocular pressure can be measured by conventional tonometric techniques.

The methods and compositions of the present invention are also intended for treatment of hypotonia and/or reduced intraocular pressure conditions of the eye. Reduced intraocular pressures are generally considered below about 8 mm Hg. Such conditions may result from a variety of causes, such as surgery for glaucoma, retinal detachment, uveitis, and the like.

The exemplified inhibitors described in detail in the Examples include cariporide, EIPA (ethylisopropylamiloride), DMA (dimethylamiloride) and amiloride, at concentrations characteristic of the NHE-1 isoform. Nevertheless, applicable compounds would include any of the beta blockers (including topical, β- and β₁-adrenergic antagonists, such as timolol), or amiloride analogs, as well as, but not limited to, the many compounds produced by Hoechst, i.e., cariporide, as well as other compounds that would be recognized as modulators of Na⁺ uptake or the anion exchange system. See, e.g., Scholz et al., Cardiovascular Research 29:260-268 (1995). Included within the families of drugs are analogs and new compounds, which represent improvements to the known compounds. Collectively, these compounds are referred to herein as the “modulating” drugs or compounds, or when combined to produce the synergistic or combined effect of the present invention, as the “modulating combination” or “modulating combined composition.”

In the present invention, a pharmaceutical composition which upon administration increases or decreases secretion of fluids into the aqueous humor as compared to the level prior to administration, is termed a “secretion modulator;” and the amount of the modulator necessary to effect the change is termed the “secretion modulating amount.” Similarly, a pharmaceutical composition which upon administration increases or decreases fluid pressure in the aqueous humor or intraocular pressure, as compared to the level prior to administration, is termed a “pressure modulator;” and the amount of the modulator necessary to effect the change is termed the “pressure modulating amount.”

In accordance with the present invention, “administration” refers to administration of the modulator to cells, e.g., the ciliary epithelial cells, in vitro or in vivo. Thus, use of the modulator composition, which can include drugs, compounds, pharmaceuticals or the like, can be used to treat an individual, such as a glaucoma patient.

Moreover, although the modulating drugs or compounds can be used alone, in the present invention they are advantageously used in combinations of two or more compounds. For example although ineffective alone, the simultaneous addition of both bumetanide and 4,4′-diisothiocyanato-stilbene-2,2′-disulfonic acid (DIDS) was shown in the examples that follow to inhibit the RVI.

Potential physiologic implications. The NHE-1 isoform of the Na⁺/H⁺ exchangers is ubiquitously expressed in all eukaryotic cells (Counillon et al., J. Biol Chem 275:1-4 (2000) and Cl⁻/HCO₃⁻ exchange is present in nearly all tissues and cells (Alper, 1994). However, such exchange can subserve intracellular pH regulation, without contributing to transepithelial transport. Data has shown that cell shrinkage can trigger uptake of solute and fluid by the PE cells (the post-RVD RVI). This fluid uptake can be inhibited by blocking the Na⁺/H⁺ antiport with dimethylamiloride or by blocking Cl⁻/HCO₃⁻ exchange by omitting CO₂/HCO₃⁻. When the Na⁺-K⁺-2Cl⁻ symport is blocked with bumetanide, the further addition of DIDS also blocks the post-RVD RVI. Thus, the paired exchange of NHE-1 and AE2 can lead to net fluid uptake from the extracellular compartment into the PE cells, as demonstrated in other systems (Jiang et al., Am J Physiol 272:C191-202 (1997)).

The presently discovered importance of the paired operation of the NHE-1 and AE2 exchangers (Na⁺/H⁺ and Cl⁻/HCO₃⁻ antiports) and the effect of blocking both, also explains the clinical efficacy of carbonic anhydrase inhibitors in treating glaucoma. Reducing the availability of H⁺ and HCO₃⁻ to both antiports, thereby synergistically inhibits the initial step in aqueous humor secretion. The current data suggest that this step could be selectively blocked in glaucomatous patients by specifically inhibiting NHE-1 with low concentrations of EIPA, DMA or cariporide, particularly in combination with bumetanide to simultaneously block the symport.

For systemic administration, the dosage of the combined agents according to this invention generally is between about 0.1 μg/kg and 10 mg/kg, preferable between 10 μg/kg and 1 mg/kg. For topical administration, dosages of between 0.000001% and 10% of the active ingredient are contemplated, preferably between about 0.1% and 4%. It will be appreciated that the actual preferred amounts of each agent will vary according to the specific agent being used, the severity of the disorder, the particular compositions being formulated, the mode of application and the species being treated. Dosages for a given host can be determined using conventional considerations, e.g., by customary comparison of the differential activities of the subject compounds in combination and of each known agent, e.g., by means of an appropriate, conventional pharmacological protocol. The agents are administered in combination or sequentially in closely timed proximity, from less than once per day (e.g., every other day) to four times per day.

Such dosages may be conveniently achieved using combined compositions having each compound present in a suitable ophthalmically-acceptable carrier or combined into a single carrier at a concentration in the range from about 0.1 weight percent to 5 weight percent. Concentrations above 5 weight percent are potentially toxic and should generally be avoided. Specific formulations are prepared in accordance with standard principles in the art, or as exemplified below.

It is also be possible to incorporate the modulating combined compounds of the present invention into controlled-release formulations and articles, where the total amount of compound is released over time, e.g., over a number of minutes or hours. Typically, the total dosage of the combined compounds will be within the limits described above for non-controlled-release formulations, but in some cases may be greater, particularly when the controlled release formulations act over relatively longer periods of time. Suitable controlled release articles for use with the compositions of the present invention include solid ocular inserts of the type available from commercial vendors.

Other controlled-release formulations may be based on polymeric carriers, including both water-soluble polymers and porous polymers having desirable controlled-release characteristics. Particularly suitable polymeric carriers include various cellulose derivatives, such as methylcellulose, sodium carboxymethylcellulose, hydroxyethylcellulose, and the like.

Suitable porous polymeric carriers can be formed as polymers and copolymers of acrylic acid, polyacrylic acids, ethylacrylates, methylnethacrylates, polyacrylamides, and the like. Certain natural biopolymers may also find use, such as gelatins, alginates, pectins, agars, starches, and the like. A wide variety of controlled-release carriers are known in the art and available for use with the present invention.

Topical compositions for delivering the modulating compounds of the present invention will typically comprise each compound present in a suitable ophthalmically acceptable carrier, or combined into a single carrier, including both organic and inorganic carriers. Exemplary ophthalmically acceptable carriers include: water, buffered aqueous solutions, isotonic mixtures of water and water-immiscible solvents, such as alkanols, arylalkanols, vegetable oils, polyalkalene glycols, petroleum-based jellies, ethyl cellulose, ethyl oleate, carboxymethylcelluloses, polyvinylpyrrolidones, isopropyl myristates, and the like. Suitable buffers include sodium chloride, sodium borate, sodium acetate, gluconates, phosphates, and the like.

The formulations of the present invention may also contain ophthalmically acceptable auxiliary components, such as emulsifiers, preservatives, wetting agents, thixotropic agents (e.g., polyethylene glycols, antimicrobials, chelating agents, and the like). Particularly suitable antimicrobial agents include quaternary ammonium compounds, benzalkonium chloride, phenylmercuric salts, thimerosal, methyl paraben, propyl paraben, benzyl alcohol, phenylethanol, sorbitan, monolaurate, triethanolamine oleate, polyoxyethylene sorbitan monopalmitylate, dioctyl sodium sulfosuccinate, monothioglycerol, and the like. Ethylenediamine tetracetic acid (EDTA) is a suitable chelating agent.

The modulating combined compounds of the present invention can be administered opthamologically, subcutaneously, intravenously, intramuscularly, topically, orally, nasally, buccally, by inhalation spray, or via an implanted reservoir. In a preferred embodiment, the therapeutic agent is administered to the eye, such as by topical administration (e.g., eye drops or emulsion). They can be administered in dosage formulations containing conventional non-toxic pharmaceutically-acceptable carriers, adjuvants and/or vehicles.

The form in which the agents are administered (e.g., capsule, tablet, solution, emulsion) will depend at least in part on the route by which they are administered. A therapeutically effective amount of the combined agent is that amount necessary to significantly reduce or eliminate symptoms associated with glaucoma, particularly to reduce or prevent elevated IOP more effectively that the effect of one of the compositions alone would have. If the effect is synergistic, the effectiveness is not only greater than that which would be achieved by each component composition acting alone, but it is also greater than that which would be expected by the simple addition of the component compositions. The therapeutically effective amount will be determined on an individual basis and will be based, at least in part, on consideration of the agent, the individual's size and gender, the severity of symptoms to be treated, the result sought. Thus, the therapeutically effective amount can be determined in light of the examples which follow by one or ordinary skill in the art, employing such factors and routine experimentation.

The therapeutically effective amount can be administered in a series of doses separated by appropriate intervals, such as hours, days or weeks, so long as the effect in the patient is that of the combined therapy. Alternatively, the therapeutically effective combined amount can be administered in a single dose. The term, “single dose,” as used herein, can be a solitary dose of the combined therapeutic compositions, and can also be a sustained release dose, such as by a controlled-release dosage formulation of a continuous infusion. The term also refers to doses of two or more drugs or therapeutic moieties that are administered sequentially, one after the other, within a brief time of less than 1 hour, preferably within less than 30 minutes, more preferably within less than 15 minutes, most preferably within 5 minutes or less—so long as the effect within the patient is that of a combined dosage, i.e., having a combined effect of the two or more drugs or therapeutic moieties being administered (having the effect of a single combined dose; i.e., an effective IOP modulating amount of the combined drugs). Other drugs, carriers, adjuvants and the like, can also be administered in conjunction with the combined agents.

The present invention is further described in the following examples. These examples are not to be construed as limiting the scope of the appended claims.

### EXAMPLES

### Example 1

### The Control of Sodium/Proton Exchangers to Control the Secretion of Excess Fluids into the Aqueous Humor

By measuring ²²Na⁺ uptake and fluorovideo-microscopy it was previously determined in U.S. S. No. 10/009,581 that PE cells possess an NHE1 Na⁺/H⁺ antiport and a Na⁺-independent Cl⁻/HCO₃⁻ exchanger which can modify intracellular pH. Volumetric measurements were also performed to confirm that these antiports could function in parallel to transfer solution from the extracellular space into the cells.

Since paired Na⁺/H⁺ and Cl⁻/HCO₃⁻ exchangers were known to contribute to the regulatory volume increase (RVI) in many other cells (Hoffmann, Curr Top Membr Transp 30:125-180 (1987)), the regulatory volume increase in the PE cells was examined. However, the secondary RVI was not observed in the PE cells at room temperature. Consequently, the volumetric experiments were conducted at 34-37° C.

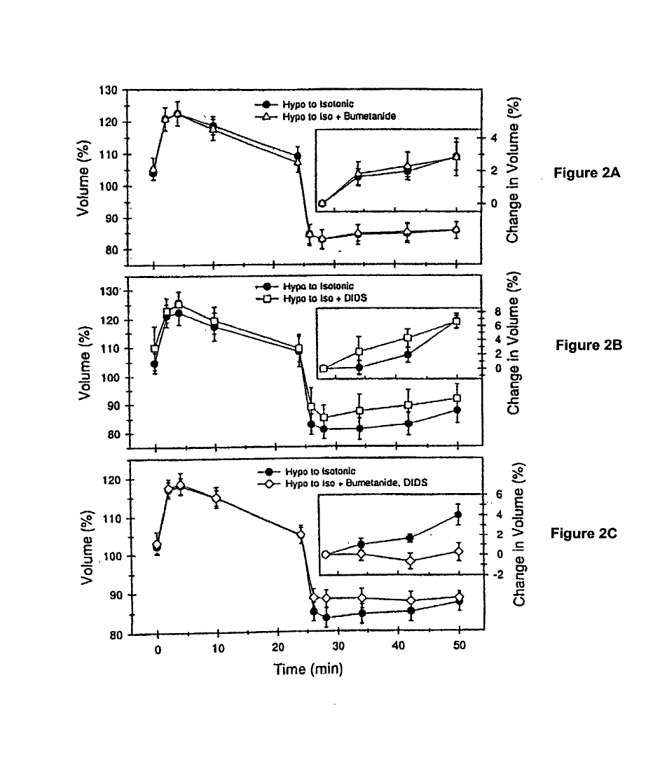

The precise time course of the baseline RVI was variable, so that the data of some experiments were better fit to an exponential expression, and others to a linear expression (filled circles, FIG. 2C). From a linear least-squares analysis, the mean±SE rate of swelling was 17.5±2.7×10⁻²%/min during the baseline post-RVD RVI, as displayed in FIG. 2. Secondary RVI was inhibited either by blocking the Na⁺/H⁺ antiport with 10 μM dimethylamiloride, or by omitting CO₂/HCO₃⁻ from the external solution.

Inhibitors were added at the same time that isotonicity was restored (t=24 min). Separate addition of either 10 μM bumetanide [to block the Na⁺-K⁺-2Cl⁻ symport (Haas et al., Am J Physiol 245:C235-240 (1983), (FIG. 2A, open triangles, N=9), or 500 μM 4,4′-diisothiocyanatostilbene-2,2′-disulfonic acid (DIDS) [to block the Cl⁻/HCO₃⁻ exchanger (Grinstein et al., J Gen Physiol 73(4):493-514 (1979))] (FIG. 2B, open squares, N=3) did not inhibit the RVI in these experiments.

However, blocking both uptake mechanisms simultaneously by addition of both bumetanide and DIDS did inhibit the RVI (FIG. 2C, open rhomboids, N=8, P<0.05). In addition, applying bumetanide alone in the nominal absence of CO₂/HCO₃⁻ was seen to produce the greatest inhibition of the regulatory volume increase (open triangles, FIG. 2A). Baseline recovery was slowed (P<0.05) and bumetanide then substantially inhibited the RVI (P<0.01).

### Example 2

### Determining the Combined Effect in Vivo of Administering Double Blocked Entry Drugs

To confirm the combined effect of blocking both known ports of NaCl entry into the ciliary epithelium (the paired antiports and the symport), the following experiments were conducted. On the assumption that if the paired activity of the antiports is blocked, the major mechanism supporting NaCl uptake from the stroma should be the Na⁺-K⁺-2Cl⁻ symport, it was predicted that bumetanide would have a substantial effect. Indeed, the same concentration of bumetanide, which was by itself ineffective, was shown to uniformly and synergistically reduce elevated mouse IOP, after either direct NHE inhibition with the acylguanidine compounds or after the carbonic anhydrase inhibitor dorzolamide.

However, first it was necessary to confirm that the paired antiports are the dominant mechanism in the first step of aqueous humor formation. Consequently, one or the other antiport was blocked to measure whether inflow, and therefore IOP, are reduced by the selected drug acting alone. Likewise, to determine whether the Na⁺-K⁺-2Cl⁻ symport played a supplemental role in supporting either uptake or release at the stromal surface, the symport was blocked by the selected inhibitor acting alone to confirm its effect on inflow. Consistent with this prediction, it was also confirmed in the mouse that bumetanide alone has no significant effect on IOP, in agreement with the earlier observation in cynomolgus monkeys.

Materials and Methods

Black Swiss outbred mice of mixed sex, 7 to 9 weeks old and approximately 30 g in weight, were obtained from Taconic, Inc. (Germantown, N.Y.). Animals were housed in accordance with National Institutes of Health recommendations, maintained under a 12-hour light-dark illumination cycle, and allowed unrestricted access to food and water. IOP measurements were performed at the same time of day (2-6 PM) to minimize diurnal effects on IOP. All procedures conformed to the ARVO Statement for the Use of Animals in Ophthalmic and Vision Research.

Before all IOP measurements, mice received general anesthesia in the form of intraperitoneal ketamine (250 mg/kg), supplemented by topical proparacaine HCl (0.5%; Allergan, Hormigueros, Puerto Rico). After reaching a stable plane of anesthesia confirmed by absent response to foot pinch, the mice were secured in a surgical stereotaxic device (David Kopf Instruments, Tujunga, Calif.), with the head positioned to avoid any pressure on the animal that could affect IOP. A heating pad at 37° C. (Delta Phase Isothermal Pad, Braintree Scientific, Braintree, Mass.) maintained body temperature. Topical proparacaine supplemented general anesthesia, and corneal dehydration was prevented by topical normal saline (309 mOsm), as necessary. The ground electrode was placed on the conjunctiva of the same or the contralateral eye, carefully avoiding any pressure on the eye.

IOP was measured with the Servo-Null Micropipette System (SNMS), an electrophysiologic, nonmanometric method of measuring pressure, previously adapted and validated for measuring IOP in the mouse (Avila et al., 2001A). The exploring, 5-μm micropipette was filled with 3 M KCl solution to ensure that the resistance of the fluid within the tip was much lower than that of the extracellular fluid. The resistance to electrical flow through the micropipette was continuously monitored and was dominated by the electrical resistance at the tip.

After entry of the tip into the anterior chamber, the step change in hydrostatic pressure forced aqueous humor into the micropipette, displacing the low-resistance 3-M KCl filling solution from the tip back toward the shank. The resultant increase in electrical resistance generated a signal to a vacuum-pressure pump that produced an equal counter-pressure that maintained the position of the aqueous humor-KCl interface at the tip of the micropipette, and thus sustains the original electrical resistance. This counter-pressure equaled the hydrostatic pressure outside the micropipette tip, in this instance the IOP. The output signal of the servo-null device (Servo-Null Micropressure System model 900A; World Precision Instruments [WPI], Sarasota, Fla.) was converted to digital form (Duo 18-Data Recording System; WPI), continuously displayed on a monitor, and saved in a computer file at three to five readings per second. Before every measurement, the system was calibrated externally against a mercury manometer in the range from 0 to 50 mm Hg at 5- to 10-mm Hg intervals.

The micropipettes were fabricated from borosilicate glass (1.5 mm outer diameter, 0.84 mm inner diameter, WPI) with a puller (Sutter Instruments, San Rafael, Calif.). The tips were beveled to an outer diameter of 5 μm and a 45° angle with a micropipette beveler (Sutter). When filled with 3 M KCl solution, these micropipettes displayed resistances of 0.25-0.60 MΩ.

Procedure for Measuring IOP

Using the SNMS as described above, the micropipette tip was next placed in the drop of proparacaine on the cornea overlying the pupil of the subject, and the output reading from the SNMS was adjusted to zero. The micropipette was then advanced across the cornea (at 20-30° to the optical axis) into the anterior chamber by a cell-penetration positioning system (model LSS 21200; Burleigh Instruments, Inc., Fishers, N.Y.) and a piezoelectric step driver (model PZ100; Burleigh). IOP was monitored after positioning the micropipette tip in the aqueous humor. The baseline IOP in the present study was 14.2±0.4 mm Hg (n=113). In measuring drug-induced changes in IOP, each animal served as its own series control. All pressures after drug application were compared with those just before the drug was added.

To determine an individual IOP reading, the mean±SEM was calculated during a 3- to 5-minute recording period. Numbers of experiments or eyes are indicated by the symbol n. The statistical significance of changes in IOP was tested with Student's paired t-test.

Drugs were applied topically in 10-μL droplets with a pipette (Eppendorf; Brinkman Instruments, Westbury, N.Y.) at the stated concentrations; total doses are also provided in parentheses. Agents were initially dissolved in dimethyl sulfoxide (DMSO). Unless otherwise stated, the final droplet solution was an isosmotic saline solution (310 mOsm) containing 1% to 8% DMSO and 0.003% benzalkonium chloride (Sigma Chemical Co., St. Louis, Mo.), commonly used to enhance ocular drug penetration. The DMSO-benzalkonium solution was found to have no effect on mouse IOP at DMSO concentrations as high as 10%. DMSO concentrations as high as 15% to 20% (Crosson, J Pharmacol. Exp. Ther. 273:320-326 (1995); Crosson, Invest. Ophthalmol. Vis. Sci. 42:1837-1840 (2001), respectively) have been reported not to alter IOP in rabbits.

Although drug concentrations in the very small volume of the mouse anterior chamber (2-4 μl) have not been definitively ascertained, comparisons of minimally effective droplet concentrations of purinergic drugs based upon their published Ki indicate that the penetrance (defined as the aqueous-to-droplet concentration ratio) is commonly approximately 1:100 to 1:1000 (Avila et al., 2001B). To extrapolate these values for purinergic drugs to the acylguanidine blockers and bumetanide is necessarily a estimate; however, this apparent penetrance of drugs in the mouse eye is not very different from the approximately 1:100 penetrance of drugs topically applied to rabbits and primates, as well.

As previously reported, changes in mouse IOP produced by this method of topical administration are mediated by local ocular, and not systemic, actions, because unilateral topical application does not alter either pupillary size (1% pilocarpine (Avila et al., 2001A), 1% tropicamide (Avila et al., Br J Pharmacol. 134:241-245 (2001B)) or IOP (100 μM adenosine Avila et al., 2001B) in the contralateral eyes. Consistent with earlier observations, the topical application of 1 mM dimethylamiloride (DMA) in this experiment did not affect the IOP of the contralateral eye (ΔIOP=0.08±0.40 mm Hg, n=6, P<0.8), but reduced IOP of the treated eye by 3.8±0.5 mm Hg (n=23, P<0.001).

Among the drugs administered were the selective Na⁺/H⁺ antiport inhibitors (direct inhibitors), dimethylamiloride (DMA) and ethylisopropylamiloride (EIPA) (Sigma Chemical Co). A third such inhibitor also used was BIIB723 (Boehringer/Ingelheim, Biberach an der Riss, Germany), which is a member of the BIIB family of Na⁺/H⁺ antiport blockers. Similar to nearly all other NHE-1 inhibitors, BIIB723 is an acylguanidine, displaying a selectivity for NHE-1 over NHE-2 of approximately 40-fold and an IC₅₀ of approximately 30 nM in cardiomyocytes and approximately 100 nM in hamster fibroblasts. The parent compound (amiloride; Merck, Rahway, N.J.) of the amiloride analogues DMA and EIPA is a low-potency inhibitor of both Na⁺/H⁺ and Na⁺/Ca²⁺ antiports and a higher-potency blocker of ENaC Na⁺ channels (Kleyman et al., J. Membr. Biol. 105:1-21 (1988)). Bumetanide (Hoffmann-La Roche, Nutley, N.J.) is a selective inhibitor of Na⁺-K⁺-2Cl⁻ co-transport. Dorzolamide (Trusopt; Merck) is a topical carbonic anhydrase inhibitor.

Results

Single Drug Effects on Mouse IOP

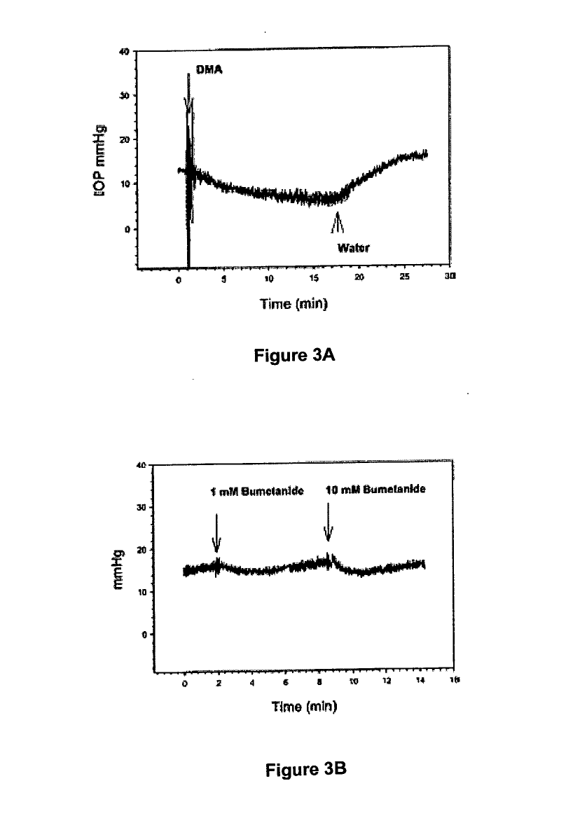

DMA, an amiloride analogue with a highly selective inhibitory effect on the NHE-1 antiport (Counillon et al., Mol. Pharmacol. 44:1041-1045 (1993)) produced a concentration-dependent lowering of IOP (FIG. 3, Table 1). Although the precise values were undefined for the threshold droplet concentrations of the drugs used, DMA was clearly effective at a droplet concentration of 1 mM (2.94 μg, n=23, Table 1), and a greater lowering of IOP (by 5.0±0.7 mm Hg) was obtained with a droplet concentration of 3 mM (8.82 μg, n=4; Table 1). Water was added at the conclusion of this and many other experiments to verify the patency of the micropipette by osmotically raising IOP.

Another amiloride analogue, EIPA, displayed the same minimally effective droplet concentration and enhanced lowering of IOP at 3 mM (300 ng; by 4.1±1.0 mm Hg, Table 1). A third acylguanidine antiport inhibitor, BIIB723, produced a maximal hypotensive effect at 3 mM (16.0 μg) of 4.9±1.7 mm Hg, similar to that of DMA (n=4, Table 1), but displayed a lower minimally effective droplet concentration (100 μM [554 ng]), n=4, Table 1). The similarity of the effects of BIIB723 at 1 mM (5.34 μg; −4.5±0.5 mm Hg) and 3 mM (16.0 μg; −4.9±1.7 mm Hg) and the similar reductions produced by all three NHE-1 inhibitors tested at 3 mM indicated that a maximal IOP reduction was achieved of 4.1 to 5.0 mm Hg. The delivered droplet concentration could not be increased in this experiment without substantially increasing the DMSO level, thereby triggering a vehicle-induced change in IOP.

TABLE 1
Single-Drug Effects of DMA, EIPA, Bumetanide, BIIB723, and Dorzolamide
on IOP.

| Drug | Class | n | Conc. | Dose | ΔIOP(mm Hg) | P |
|---|---|---|---|---|---|---|
| DMA | Na/H antiport inhibitor | 3 | 100 μM | 294 ng | +0.9 ± 0.9 | |
| | | 23 | 1 mM | 2.94 μg | −3.8 ± 0.5 | <0.001 |
| | | 4 | 3 mM | 8.82 μg | −5.0 ± 0.7 | <0.01 |
| EIPA | Na/H antiport inhibitor | 3 | 100 μM | 300 ng | +0.8 ± 0.2 | |
| | | 10 | 1 mM | 3.00 μg | −2.6 ± 0.5 | <0.001 |
| | | 6 | 3 mM | 9.00 μg | −4.1 ± 1.0 | <0.01 |
| BIIB | Na/H antiport inhibitor | 3 | 10 μM | 53.4 ng | −0.4 ± 1.9 | |
| | | 4 | 100 μM | 534 ng | −2.7 ± 0.4 | <0.01 |
| | | 17 | 1 mM | 5.34 μg | −4.5 ± 0.5 | <0.001 |
| | | 4 | 3 mM | 16.0 μg | −4.9 ± 1.7 | |
| Dorzolamide | CA topical inhibitor | 11 | 55.4 mM | 200 μg | −2.9 ± 0.6 | <0.001 |
| Bumetanide | Na-K-2Cl symport | 4 | 10 μM | 36.4 ng | −0.2_1.6 | |
| | blocker | 3 | 100 μM | 364 ng | −0.8 ± 0.7 | |
| | | 7 | 1 mM | 3.64 μg | −0.7 ± 1.6 | |
| | | 12 | 10 mM | 36.4 μg | −1.2 ± 0.6 | |
| Contra-lateral | | 6 | 1 mM | 2.94 μg | +0.1 ± 0.4 | |
| Drugs | | | | | | |
| DMA | | | | | | |
| Vehicle | | 5 | 10% | 10.0 μg | −0.3 ± 0.6 | |
| DMSO | | | | | | |
| (10%) | | | | | | |

Conc. = concentration.

By the aqueous-to-droplet concentration ratio discussed above, the minimally effective droplet concentration of 1 mM for DMA and EIPA (Table 1) appears to correspond to approximately 1 to 10 μM in the aqueous humor, and the minimally effective droplet concentration of 100 μM for BIIB723 corresponded to aqueous humor concentrations of ˜0.1 to 1 μM. The differences may arise from a higher penetrance for BIIB723, because the IC₅₀ observed for this drug (30-100 mM, unpublished results) is similar to that of EIPA (50 nM, Scholz et al., Cardiovasc. Res. 29:260-268 (1995)). Although BIIB723 may penetrate more effectively than DMA or EIPA, it is likely that all three NHE-1 inhibitors exerted a maximal effect at 3 mM (as discussed in the Results, below), uniformly reducing IOP by 4.1 to 5.0 mm Hg.

Carbonic anhydrase inhibition reduces the rate of production of H⁺ and HCO₃⁻, which in turn must slow the rate of delivery of H⁺ and HCO₃⁻ to all cell sites, including the antiports. Recognizing that inhibiting carbonic anhydrase with intraperitoneal acetazolamide lowers mouse IOP (by 11.9±1.3 mm Hg) (Avila et al., 2001A), it was found that topical application of dorzolamide also reduces IOP, albeit to a lesser extent at the droplet concentrations applied (Table 1). Amiloride, which inhibits NHE-1 antiports at a potency 1 to 2 orders of magnitude lower than the amiloride analogues DMA and EIPA (Counillon et al., 2000), itself exerted no significant effect on mouse IOP at a droplet concentration of 1 mM (2.30 μg, n=7, data not shown). To reach a 10-mM concentration, it was necessary to solubilize the amiloride in 30% DMSO. After pretreatment with vehicle containing 30% DMSO, subsequent application of 10 mM amiloride in the same concentration of vehicle did not alter that IOP (ΔIOP=1.0±0.7 mm Hg, n=4, P>0.2). Thus, at a concentration 10 times higher than EIPA's minimal effective concentration, amiloride had no effect, consistent with the known ratio of the potency of these inhibitors (3.9:0.07 μM, or ˜56) when applied to PE cells.

In contrast to the IOP reductions triggered by the three selective inhibitors of the NHE-1 antiport at droplet concentrations of 0.1 to 3 mM (Table 1), blockage of the Na⁺-K⁺-2Cl⁻ symport with droplet concentrations of 0.1 to 10 mM (364 ng to 36.4 μg) bumetanide, had no significant effect on IOP (FIG. 3, Table 1).

Combined Drug Effects on Mouse IOP

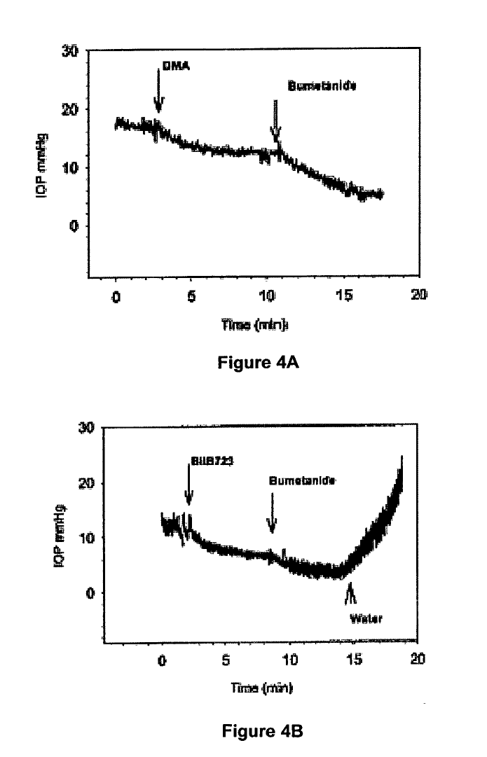

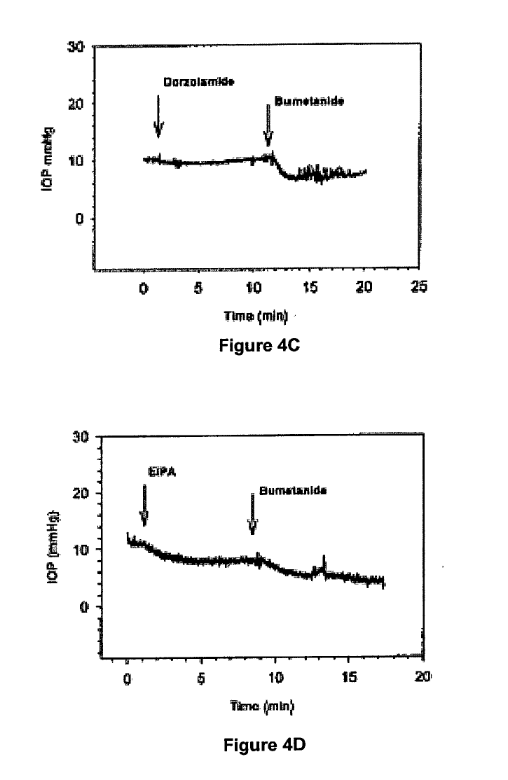

Electron microprobe analyses have suggested that inhibition of the Na⁺-K⁺-2Cl⁻ symport lowers Cl⁻ uptake by the ciliary epithelium under conditions in which the turnover rate of the Na⁺/H⁺ antiport is reduced. To test this hypothesis in vivo, bumetanide was applied after first reducing Na⁺/H⁺ antiport exchange either directly with acylguanidine inhibitors or indirectly with a carbonic anhydrase inhibitor (FIG. 4, Table 2). In each case, topical application of the first drug produced the anticipated significant decrease in IOP. Thereafter, the same 10-mM droplet concentration (36.4 μg) of bumetanide, which was ineffective by itself, triggered significant further lowering of IOP.

The entries in Table 2 present the changes in IOP produced first (1^{st}) by the initial drug (with respect to baseline) and second (2^{nd}) by the later addition of bumetanide (in comparison with the previous experimental period). In every case, the secondary application of bumetanide reduced IOP by 3.8 to 4.0 mm Hg (Table 2). Directly inhibiting the Na⁺/H⁺ antiport with a submaximal 1-mM concentration (5.34 μg) of BIIB723 slightly enhanced the reduction in IOP previously triggered by indirectly inhibiting the antiport with dorzolamide (ΔIOP=−0.7±0.2 mm Hg, Table 2).

TABLE 2
Effects on IOP of Combined (Sequential) Medications

| | | | ΔIOP(mm | | ΔIOP(mm | |
|---|---|---|---|---|---|---|
| 1st Drug/ | | 1st Drug Conc./ | Hg) after | | Hg) (after 1st | |
| 2nd Drug | n | 2nd Drug Conc. | base | P | drug) | P |
| Dorzolamide (CA inhibitor)/ | 4 | 55.5 mM(200 μg)/ | −2.0 ± 0.4 | <0.05 | | |
| Bumetanide (symport | | 10 mM (36.4 μg)/ | | | −3.9 ± 1.0 | <0.05 |
| inhibitor) | | | | | | |
| BIIB (Na⁺/H⁺ antiport | 6 | 1 mM (5.34 μg)/ | −2.9 ± 1.0 | <0.05 | | |
| inhibitor / | | | | | | |
| Bumetanide (symport | | 10 mM (36.4 μg) | | | −3.9 ± 0.9 | <0.01 |
| inhibitor) | | | | | | |
| DMA (Na⁺/H⁺ | 6 | 1 mM (2.94 μg)/ | −4.0 ± 0.8 | <0.01 | | |
| antiport inhibitor) / | | | | | | |
| Bumetanide (symport | | 10 mM (36.4 μg) | | | −3.8 ± 0.7 | <0.01 |
| inhibitor) | | | | | | |
| EIPA (Na⁺/H⁺ | 6 | 1 mM (3.00 μg) / | −2.4 ± 0.6 | <0.01 | | |
| antiport inhibitor) / | | | | | | |
| Bumetanide (symport | | 10 mM (36.4 μg) | | | −4.0 ± 0.6 | <0.01 |
| inhibitor) | | | | | | |
| Dorzolamide (CA inhibitor) / | 7 | 55.4 mM(200 μg)/ | −3.5 ± 0.9 | <0.01 | | |
| BIIB (Na⁺/H⁺ | | 1 mM (5.34 μg) | | | −0.7 ± 0.2 | <0.01 |
| antiport inhibitor) | | | | | | |

Conc. = concentration.

In sum, these salient findings demonstrate that inhibitors of the NHE-1 Na⁺/H⁺ antiport reduced IOP at 1-mM droplet concentrations, but the far less potent parent compound (amiloride) had no effect on IOP at tenfold higher concentration. Topical application of the carbonic anhydrase inhibitor dorzolamide reduced IOP in the mouse. Similarly, application of a selective Na⁺-K⁺-2Cl⁻ symport inhibitor (bumetanide) itself had no significant effect. However, after first inhibiting the NHE antiports, either directly with acylguanidine blockers or indirectly with dorzolamide, the subsequent application of bumetanide triggered a highly significant further synergistic reduction in IOP of 3.8 to 4.0 mm Hg.

### Example 3

### Determining the Combined Effect in Vivo of Selective Blocking of Entry and Release Steps in Aqueous Humor Formation

The following work is designed to more effectively control IOP by selectively and simultaneously (or by producing a combined effect in the patient) blocking both (1) the first step of aqueous humor formation (entry into the ciliary epithelium), and (2) the release step of Cl⁻ from the aqueous humor. As discussed with regard to the entry step above, the NHE-1 exchanger can be selectively blocked or inhibited, which is important in the first step of aqueous humor formation. However, it is also possible to block activation of the final step of aqueous humor formation by applying, e.g., A₃-subtype adenosine-receptor antagonists.

Based upon the findings from the inventors' laboratory, as reported by Avila et al., in Invest. Ophthalmol. Vis. Sci., 43: in press (2002) (herein incorporated by reference) IOP was monitored using the SNMS method and analysis procedures as described in Example 2, to demonstrate the effect of blocking both entry and exit steps of aqueous humor formation in a test animal, an A₃AR-knockout mice. The observations that A₃AR agonists activate Cl⁻ channel led to the hypothesis that these agonists would increase aqueous humor secretion and thereby IOP in vivo, and that A₃AR antagonists would exert the opposite effects. In the absence of the A₃-subtype adenosine receptor, reduced baseline activity of the NPE Cl⁻ channels was expected, thereby reducing both inflow and IOP.

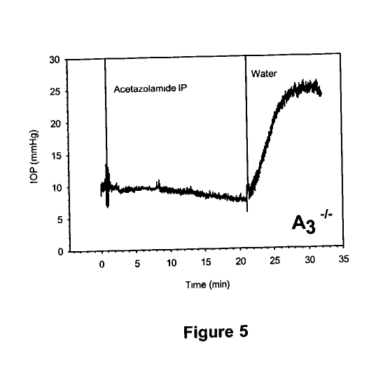

Indeed, in black Swiss outbred mice, IOP was significantly lower in A₃AR^{-/-} knockout mice (12.9±0.7 mm Hg; n=44 eyes) as compared with matched, normal, control animals (17.4±0.6 mm Hg). Even when the IOP of an A₃AR-knockout mouse was as low as 10 mm Hg because of reducing the rate of the exit step (FIG. 5), blocking the entry step (with acetazolamide), reduced IOP even further by 2-3 mm Hg. The intraocular pressure cannot fall below the episcleral venous pressure, which in humans has been estimated to be 8.0-11.5 mm Hg. Thus, the combined approach of blocking both the entry step (by inhibiting the paired antiports) and the exit step (by applying A₃-subtype adenosine receptor antagonists) maximizes the effect and produces the lowest possible reduction in IOP.

A combinatorial strategy similar to that described for the double blockade of the entry step (Example 2) is used for the combined blocking of the enter and exit steps, in which drops will be initially applied to the subject eye, containing 25 μM MRS 1191 (a selective antagonist of the A₃ARs). Then, either in the same drops, or in drops applied immediately thereafter so as to achieve a combined effect in the subject, topical antiport blockers (such as, e.g., 1 mM DMA or 1 mM EIPA) are directly or indirectly applied to achieve optimal reduction of IOP in the subject.

Accordingly, it is shown in light of the foregoing, in light of in vivo evidence, that selected combinations of drugs or therapeutic moieties (in combination referred to as the “combined modulator”) effectively and synergistically lower IOP by: (1) double-blocking of uptake step, wherein both transporters in the first (entry step) of aqueous humor formation are blocked or inhibited; or (2) blocking of the entry and exit steps, wherein the sodium-hydrogen (Na/H) exchanger underlying the entry step is blocked or inhibited, and also the activity is lowered or reduced of the chloride (Cl⁻) channels involved in the second (exit) step of aqueous humor formation.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method for regulating, controlling or modulating aqueous humor secretion, comprising the step of administering to ciliary epithelial cells of the aqueous humor, an effective secretion-modulating amount of a combined modulator, which is, or forms, a combination of pharmaceutical compositions comprising an effective secretion-modulating amount of a modulator of one or more antiports and a modulator of one or more symports.

2. The method of claim 1, wherein the one or more antiports are selected from the group consisting of a Na⁺/H⁺ exchanger or a Cl⁻/HCO₃⁻ exchanger.

3. The method of claim 1, wherein the one or more antiports are selected from the group consisting of a Na⁺/H⁺ exchanger and a Cl⁻/HCO₃⁻ exchanger.

4. The method of claim 1, wherein both transporters in the entry step of aqueous humor formation (the paired Na⁺/H⁺ and Cl⁻/HCO₃⁻ antiports and the Na⁺-K⁺-2Cl⁻ symport) are blocked.

5. The method of claim 1, wherein secretion in the aqueous humor cells is elevated, and wherein the combined modulator is administered in an amount sufficient to reduce the elevated secretion.

6. The method of claim 1, wherein the method of regulating, controlling or modulating aqueous humor secretion further comprises regulating, controlling or modulating fluid pressure in the aqueous humor ciliary epithelial cells.

7. The method of claim 6, wherein the fluid pressure is elevated, and wherein the combined modulator is administered in an amount sufficient to reduce the elevated pressure.

8. The method of claim 1, wherein the Na⁺/H⁺ exchange occurs at the NHE-1 antiport.

9. The method of claim 1, wherein the Cl⁻/HCO₃⁻ exchange occurs at the AE2 antiport.

10. The method of claim 1, wherein the modulating effect is reversible upon cessation of administration of the combined modulator.

11. The method of claim 1, wherein the combined modulator is administered to the cells in vitro.

12. The method of claim 1, wherein the combined modulator is administered to the cells in vivo.

13. The method of claim 12, wherein the modulating effect occurs in the formation of the aqueous humor of a human patient, comprising the step of administering to the patient an effective intraocular pressure-modulating amount of the combined modulator.

14. The method of claim 1, wherein the pharmaceutical compositions forming the combined modulator are administered simultaneously.

15. The method of claim 1, wherein the pharmaceutical compositions forming the combined modulator are administered sequentially in any order, such that together a combined effect is achieved in the ciliary epithelial cells.

16. The method of claim 1, wherein the regulating, controlling or modulating effect of administering the combined modulator on aqueous humor formation is synergistic, as compared with an additive combination of the independent pharmaceutical compositions forming the combined modulator.

17. The combined modulator used to achieve the regulating, controlling or modulating effect in accordance with claim 1.

18. A method for regulating, controlling or modulating aqueous humor secretion, comprising the step of administering to ciliary epithelial cells of the aqueous humor, an effective secretion-modulating amount of a combined modulator which is, or forms, a combination of pharmaceutical compositions comprising at least one modulator that blocks or inhibits at least one entry step in the formation of the aqueous humor and at least one modulator that activity is lowers or reduces the activity of at least one exit step in the formation of the aqueous humor.

19. The method of claim 18, wherein a sodium-hydrogen (Na⁺/H⁺) exchanger underlies the entry step being blocked.

20. The method of claim 18, wherein chloride (Cl⁻) channels activity, involved in the exit step of aqueous humor formation, is lowered or reduced.

21. The method of claim 18, wherein secretion in the aqueous humor cells is elevated, and wherein the combined modulator is administered in an amount sufficient to reduce the elevated secretion.

22. The method of claim 18, wherein the method of regulating, controlling or modulating aqueous humor secretion, further comprises regulating, controlling or modulating fluid pressure in the aqueous humor ciliary epithelial cells.

23. The method of claim 22, wherein the fluid pressure is elevated, and wherein the combined modulator is administered in an amount sufficient to reduce the elevated pressure.

24. The method of claim 18, wherein the modulating effect is reversible upon cessation of administration of the combined modulator.

25. The method of claim 18, wherein the combined modulator is administered to the cells in vitro.

26. The method of claim 18, wherein the combined modulator is administered to the cells in vivo.

27. The method of claim 26, wherein the modulating effect occurs in the formation of the aqueous humor of a human patient, comprising the step of administering to the patient an effective intraocular pressure-modulating amount of the combined modulator.

28. The method of claim 18, wherein the pharmaceutical compositions forming the combined modulator are administered simultaneously.

29. The method of claim 18, wherein the pharmaceutical compositions forming the combined modulator are administered sequentially in any order, such that together a combined effect is achieved in the ciliary epithelial cells.

30. The method of claim 18, wherein the regulating, controlling or modulating effect of administering the combined modulator on aqueous humor formation is synergistic, as compared with an additive combination of the independent pharmaceutical compositions forming the combined modulator.

31. The combined modulator used to achieve the regulating, controlling or modulating effect in accordance with claim 18.
